SEQUENCE LISTING (39 sequences)

SEQ ID NO: 1 - DNA, 21 bp, Artificial Sequence
  Synthetic oligonucleotide probe
anaataaggagcggacagttt

SEQ ID NO: 2 - DNA, 22 bp, Artificial Sequence
  Synthetic oligonucleotide probe
anaataaggaacggacaggttt

SEQ ID NO: 3 - DNA, 35 bp, Artificial Sequence
  Synthetic oligonucleotide probe
anactttccaatgangttcctgttttatggacttt

SEQ ID NO: 4 - DNA, 40 bp, Artificial Sequence
  Synthetic oligonucleotide probe
anagtccctagacganaatgtctaatcttcatcaggatna

SEQ ID NO: 5 - DNA, 39 bp, Artificial Sequence
  Synthetic oligonucleotide probe
anatccctagacganaatgtctaatcttcatcaggatna

SEQ ID NO: 6 - DNA, 24 bp, Artificial Sequence
  Synthetic oligonucleotide probe
ttnatcctttccgattagttganc

SEQ ID NO: 7 - DNA, 32 bp, Artificial Sequence
  Synthetic oligonucleotide probe
tgggaccancttgacgagactagtaccacanc

SEQ ID NO: 8 - DNA, 30 bp, Artificial Sequence
  Synthetic oligonucleotide probe
tanagagtgacttgggggtttgtctggana

SEQ ID NO: 9 - DNA, 34 bp, Artificial Sequence
  Synthetic oligonucleotide probe
tctggaccttanactttgattctgttttatacna

SEQ ID NO: 10 - DNA, 32 bp, Artificial Sequence
  Synthetic oligonucleotide probe
tanatctgaacggangccgtcactaccatgac

SEQ ID NO: 11 - DNA, 32 bp, Artificial Sequence
  Synthetic oligonucleotide probe
tantagtgtgaccacgatttttcctgatganc

SEQ ID NO: 12 - DNA, 35 bp, Artificial Sequence
  Synthetic oligonucleotide probe
tantgacaagagaacttcctttacggggtaatana

SEQ ID NO: 13 - DNA, 17 bp, Artificial Sequence
  Synthetic oligonucleotide probe
anaaggagcggacaggt

SEQ ID NO: 14 - DNA, 24 bp, Artificial Sequence
  Synthetic oligonucleotide probe
anaagatactagtactctcagttt

SEQ ID NO: 15 - DNA, 24 bp, Artificial Sequence
  Synthetic oligonucleotide probe
anaagatactactactctcagttt

SEQ ID NO: 16 - DNA, 43 bp, Artificial Sequence
  Synthetic oligonucleotide probe
anacttcganacccgatgcacctactggtcgacaagcacattt

SEQ ID NO: 17 - DNA, 45 bp, Artificial Sequence
  Synthetic oligonucleotide probe
anacggcacacctcggggcttgaggtacccanaggtcatcttana

SEQ ID NO: 18 - DNA, 31 bp, Artificial Sequence
  Synthetic oligonucleotide probe
tggtctanaccgacgtcgactcagtctcana

SEQ ID NO: 19 - DNA, 26 bp, Artificial Sequence
  Synthetic oligonucleotide probe
tcccaccctagtgtacangtgacanc

SEQ ID NO: 20 - DNA, 32 bp, Artificial Sequence
  Synthetic oligonucleotide probe
tgttcccatanacctctcccccggagtggang

SEQ ID NO: 21 - DNA, 33 bp, Artificial Sequence
  Synthetic oligonucleotide probe
tanaacaccctcgtcccttctcccttccttana

SEQ ID NO: 22 - DNA, 27 bp, Artificial Sequence
  Synthetic oligonucleotide probe
tanaccaacgtcanttgttccgacccc

SEQ ID NO: 23 - DNA, 29 bp, Artificial Sequence
  Synthetic oligonucleotide probe
tancccctaccacctttatccctggatna

SEQ ID NO: 24 - DNA, 26 bp, Artificial Sequence
  Synthetic oligonucleotide probe
tangtgtgagagacgtgatggagang

SEQ ID NO: 25 - DNA, 22 bp, Artificial Sequence
  Synthetic oligonucleotide probe
anaatatgcacggtccaccttt

SEQ ID NO: 26 - DNA, 22 bp, Artificial Sequence
  Synthetic oligonucleotide probe
anaatatgcatggtccaccttt

SEQ ID NO: 27 - DNA, 34 bp, Artificial Sequence
  Synthetic oligonucleotide probe
gnaccganatgggggaccccttctcgtctctttt

SEQ ID NO: 28 - DNA, 39 bp, Artificial Sequence
  Synthetic oligonucleotide probe
anatcgtgggtccggacctagtcggggagtnacactana

SEQ ID NO: 29 - DNA, 35 bp, Artificial Sequence
  Synthetic oligonucleotide probe
tantctcaggttagaatcctgtgttttaccacana

SEQ ID NO: 30 - DNA, 31 bp, Artificial Sequence
  Synthetic oligonucleotide probe
tcatcgaacaaaaanagacttttcccatana

SEQ ID NO: 31 - DNA, 33 bp, Artificial Sequence
  Synthetic oligonucleotide probe
tanggaggttggatatcttccttcactttcang

SEQ ID NO: 32 - DNA, 31 bp, Artificial Sequence
  Synthetic oligonucleotide probe
tggaaggaganaggacagttcacggaggana

SEQ ID NO: 33 - DNA, 32 bp, Artificial Sequence
  Synthetic oligonucleotide probe
tgtgtagtacactggagangtcactggtgana

SEQ ID NO: 34 - DNA, 31 bp, Artificial Sequence
  Synthetic oligonucleotide probe
tanagcccggancttgatgatgggggtcttg

SEQ ID NO: 35 - DNA, 31 bp, Artificial Sequence
  Synthetic oligonucleotide probe
tcccaccgttagtttccganattgaacgana

SEQ ID NO: 36 - DNA, 31 bp, Artificial Sequence
  Synthetic oligonucleotide probe
tantaccgtcactctactcctagacgagana

SEQ ID NO: 37 - DNA, 30 bp, Artificial Sequence
  Synthetic oligonucleotide probe
tancccccanctctcctcacggactcctcc

SEQ ID NO: 38 - DNA, 35 bp, Artificial Sequence
  Synthetic oligonucleotide probe
tanactgactactctcggtcctcgactcttttana

SEQ ID NO: 39 - DNA, 31 bp, Artificial Sequence
  Synthetic oligonucleotide probe
tangcttggatttctgcanaacgggttaccc

(19) United States
(12) Patent Application Publication
(10) Pub. No.: US 2004/0152118 A1
(43) Pub. Date: Aug. 5, 2004

(54) Methods and compositions for detecting nucleic acid sequences

(76) Inventors: Reuel B. Van Atta, Mountain View, CA (US); Michael L. Wood, Mountain View, CA (US)

Correspondence Address: RALPH T. LILORE, 371 FRANKLIN AVENUE, THIRD FLOOR - PO BOX 570, NUTLEY, NJ, 07110, US

(21) Appl. No.: 10/766,266

(22) Filed: Jan. 27, 2004

Related U.S. Application Data
(60) Provisional application No. 60/443,820, filed on Jan. 29, 2003.

(51) Int. Cl. C12Q 1/68; C07H 21/04

(52) U.S. Cl. 435/6; 536/24.3

(57) ABSTRACT

Methods and compositions are provided for the detection of polymorphisms utilizing pairs or sets of nucleic acid probes capable of crosslinking to: (1) the target sequence via the formation of probe-target hybridization complexes; and/or (2) each other via the formation of probe-probe stem structures.

### TECHNICAL FIELD

The field of this invention is nucleic acid sequence detection, and more specifically, the detection of polymorphic sequences of interest utilizing a crosslinking probe system.

### BACKGROUND

Genetic polymorphisms, or variations in DNA sequences, are widespread throughout the human genome and contribute to variations in protein function among individuals, which can lead to disease conditions. There are already a substantial number of genes, which when mutated, are known to be associated with various diseases in humans, including cystic fibrosis, Huntington's disease, beta thalassemia, and sickle cell anemia. In some instances, such as sickle cell anemia, there is a specific point mutation in a gene that causes the disease. In other cases, such as cystic fibrosis, there are numerous point mutations spread throughout the genes that are associated with the disease.

In addition to the identification of disease-associated genes, there is also increasing interest in pharmacogenetics, which attempts to harness rapidly-expanding genetic knowledge to identify genes involved in variable responses to drugs. Drug responsiveness can range from subtherapeutic and ineffective dosing at one extreme to toxic and potentially lethal overdosing at the other. There is a growing body of evidence indicating that this variation may be due at least in part to genetic factors, including polymorphisms within one or more genes coding for proteins involved in critical metabolic and/or physiological pathways relevant to drug action. Understanding the genetic foundations of the pharmacokinetic and pharmacodynamic factors involved in drug variability among individuals will enable truly personalized medicine, in which patients can be screened in advance to ensure proper drug selection and dosing based on their own genetic makeup, thereby maximizing responsiveness and minimizing toxicity.

Conventional screening methods for detecting such polymorphisms typically include a target amplification step. Importantly, however, it is critical that the amplification process be highly specific, since the amplification of untargeted sequences along with the desired target impairs significantly the reliability of the assay. Typically, this amplification step is achieved using the polymerase chain reaction (PCR). (See Saiki et al., Science 1988; 239(4839):487-491 and Mullis et al., U.S. Pat. No. 4,683,195). A major problem with PCR is non-specific target amplification, which often leads to increased background signals owing to spurious signal generation.

Backman et al., EP 320 308, disclose an alternative method, known as the ligase chain reaction (LCR), for amplifying a target nucleic acid sequence. Unfortunately, as with PCR, a major problem associated with LCR is the production of undesirable background signal. This undesirable effect is caused by a phenomenon whereby the oligonucleotide pair that is complementary to the target ligates to each other even in the absence of the target in the sample. These target-independent ligation products, which can in turn serve as templates for the production of more target-independent products in subsequent rounds of amplification, can then compromise the reliability of LCR-based assays.

Both PCR and LCR have additional drawbacks due to the requirement of polymerases or ligases in order to achieve target amplification. In addition to being expensive, such enzymes exhibit lot-to-lot variations in activity and in the presence of undesired nuclease contaminants. Such variations detract further from the reliability of PCR- and LCR-based methods.

Previous improvements to nucleic acid polymorphism detection have been introduced in U.S. Pat. Nos. 6,277,570 and 6,495,676, which disclose an elegant system employing capture probes capable of hybridizing with and crosslinking to a target sequence of interest. The covalent bonds formed during the crosslinking step enable the application of high-stringency washes to reduce the occurrence of background signals. Although these systems improve greatly upon previous detection systems, they may not be appropriate in every instance.

For example, the aforementioned assays require the presence of a crosslinking site (typically a thymine base) near the mutation site in the target sequence. Furthermore, they require the target strand remains intact after crosslinking to the extent that the target associates a capture probe with one or more reporter probes irreversibly through covalent linkages between it and the probes. This latter requirement can be particularly problematic if the target nucleic acid is RNA. What is needed, therefore, are improved compositions and methods for detecting target sequences that do not include an amplification step, and that further reduce spurious background signals, thereby leading to the development of more reliable target sequence detection assays.

### RELEVANT LITERATURE

Nucleic acid crosslinking probes for DNA/RNA diagnostics are disclosed in Wood et al., Clin. Chem. 1996; 42(S6):S196. Crosslinker-containing probes capable of discriminating between single-base polymorphic sites in target sequences in solution-based hybridization assays have been reported in Zehnder et al., Clin. Chem. 1997; 43(9):1703-1708.

U.S. Pat. Nos. 5,616,464; 5,767,259; 6,004,513; and 6,177,243 describe a nucleic acid amplification system using probes with side chain structures capable of crosslinking with each other.

### SUMMARY OF INVENTION

The present invention provides improved compositions and methods for detecting genetic polymorphisms of interest. In one embodiment, the invention provides a nucleic acid probe comprising a target complementary sequence and a side chain, wherein the target complementary sequence and the side chain each comprises at least one crosslinking agent.

In a preferred embodiment, the invention provides a pair of nucleic acid probes. The first probe of the pair comprises a first side chain, and the second probe of the pair comprises a second side chain, wherein at least one of the first or second side chains comprises at least one crosslinking agent. The first probe and second probe further comprise a first target complementary sequence and a second target complementary sequence, respectively, wherein at least one of the first target complementary sequence and the second target complementary sequence comprises at least one crosslinking agent. In this embodiment, the first target complementary sequence and the second target complementary sequence are capable of hybridizing to adjacent portions of the target sequence of interest, such that the first and second side chains hybridize to form a stem upon hybridization of the probes to the target sequence.

Further, upon hybridization of the probe pair to the target sequence, the crosslinking agent may then be activated so as to crosslink the probe pair to each other and to the target sequence of interest, if present in a sample. Thus, in a further preferred embodiment, the invention comprises a nucleic acid probe pair having the structure described above crosslinked to each other via a stem formed by the first and second side chains of said first and second probes, respectively, and further crosslinked to the target sequence of interest.

In another embodiment, the invention provides a method for detecting a target nucleic acid sequence in a sample. The method comprises combining the sample with at least one pair of the above-described probes under conditions of base pairing between the probes and the target nucleic acid to produce an assay medium. Upon hybridization of the probe pair to adjacent portions of the target sequence, the first and second side chains are brought into proximity and non-covalently associate to form a stem. The crosslinking agent is then activated, whereby a covalent crosslink forms between the side chain members of the stem, and between the target complementary sequence(s) and the target sequence. Finally, the presence of crosslinked probes, which is indicative of the presence of the target sequence in the sample, is detected.

In another preferred embodiment, the invention provides a nucleic acid probe set comprising at least one capture probe and at least two flanking probes. The first flanking probe comprises a first targeting sequence substantially complementary to a first portion of the target sequence of interest, and a first side chain. The capture probe comprises a second targeting sequence substantially complementary to a second portion of a target sequence of interest, a second side chain having a sequence substantially complementary to the sequence of the first side chain, and a third side chain. The second flanking probe comprises a third targeting sequence substantially complementary to a third portion of the target sequence, and a fourth side chain having a sequence substantially complementary to the sequence of the third side chain. Preferably, at least one of the first, second, and third targeting sequences further comprises at least one crosslinking agent; at least one of the first and second side chains further comprises at least one crosslinking agent; and, at least one of the third and fourth side chains further comprises at least one crosslinking agent.

In another embodiment, the invention provides a method for genotyping a target sequence in a sample. The target sequence comprises an interrogation region comprising an interrogation position, and the method comprises hybridizing the interrogation region to a crosslinkable probe mixture to form a hybridization complex. The crosslinkable probe mixture comprises a nucleic acid probe pair or a nucleic acid probe set as described above, wherein the target complementary sequence of at least one probe of the pair or set comprises a detection position perfectly complementary to the interrogation position of a desired allele, and further comprises a sequence substantially complementary to the sequence upstream and downstream of the interrogation position in the interrogation region. In a preferred embodiment, the second targeting sequence of the probe set comprises the detection position, and the corresponding interrogation position is located within the second portion of the target sequence.

Upon activation of the crosslinking agent, the probe containing the detection position becomes covalently crosslinked either directly to the target and/or indirectly, for example through one or more flanking probes that crosslink to both the probe containing the detection position and to the target. The resulting crosslinked hybridization complex, which contains the target, is washed at least once under high-stringency conditions. Finally, the presence of the target is detected using, for example, a reporter moiety that is attached to at least one of the probes in the probe mixture. In a preferred embodiment, one or more reporter probes, each containing one or more reporter moieties, are included to the total ensemble of probes used to perform an assay. These reporter probes are generally designed to hybridize and crosslink to regions in the target sequence that do not overlap with the first, second, or third portion of the target sequence.

### DESCRIPTION OF THE FIGURES

FIG. 1 illustrates one embodiment of a nucleic acid probe pair according to the present invention. M and T denote a mutation site and thymine, respectively on a target nucleic acid strand. Z and W denote coumarin-based (crosslinking) nucleotide analogs, whereas X and Y denote natural and/or unnatural nucleotides.

FIG. 2 illustrates one embodiment of a nucleic acid probe set according to the present invention, with the designations as identified above and with U and V denoting natural and/or unnatural nucleotides.

FIG. 3 illustrates a schematic representation of the nucleic acid probe set described in Example 1.

### DETAILED DESCRIPTION OF INVENTION

The present invention provides compositions and methods for detecting one or more types of polymorphisms that may be present in a gene or genes of interest. The polymorphism may be either inherited or spontaneous, germline or somatic, or a marker of interspecies variation. Polymorphisms or mutations of interest include single nucleotide polymorphisms (SNPs), as well as those related to gene dosage abnormalities such as deletions and duplications, substitutions, insertions, translocations, rearrangements, variable number of tandem repeats, short tandem repeats, retrotransposons such as Alu and long interspersed nuclear elements, and the like. By convention, sequence variants present at frequencies less than 1% are generally considered mutations, whereas those present at higher frequencies are considered polymorphisms. As used herein, the term “polymorphism” means any DNA sequence variation of any type or frequency.

In a preferred embodiment, the invention provides a composition for detecting one or more SNPs in a sample for one or more genes of interest. Preferably, the composition comprises either a pair of probes or a set of three probes having targeting sequences substantially complementary to adjacent portions of the target sequence, and side chains capable of associating to form one or more stems when the targeting sequences are hybridized to the respective portions of the target sequence. At least one of the targeting sequences in at least one probe of the pair or set comprises a detection position complementary to an interrogation position defining the SNP of interest.

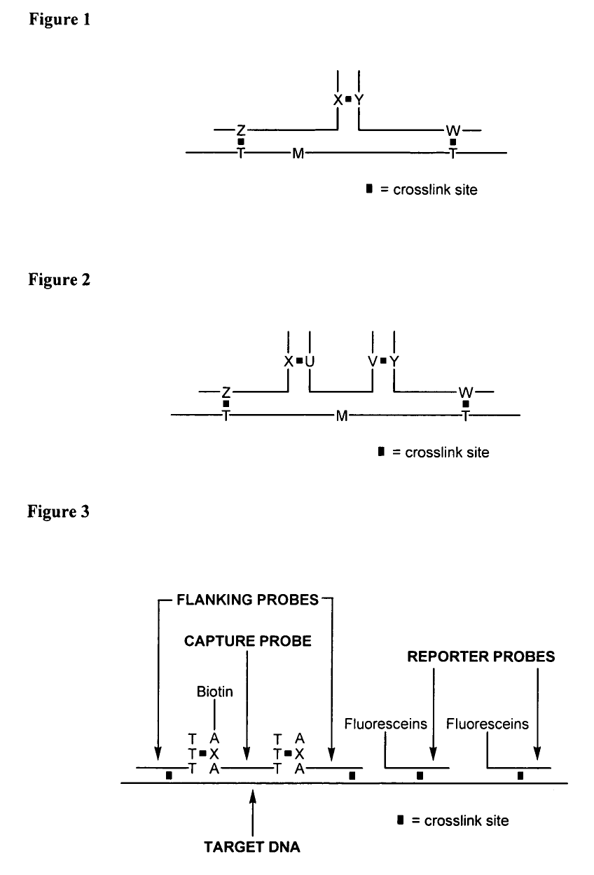

In one aspect, illustrated in FIG. 1, a pair of probes is provided having first and second targeting sequences, at least one of said first or second targeting sequences comprising at least one crosslinking agent for crosslinking the probe to the target sequence. In accordance with the present invention, at least one of the side chains of the probe pair also comprises at least one crosslinking agent such that the side chains may also be crosslinked together to form a crosslinked stem. The presence of the crosslinked stem in accordance with the present invention obviates the need for the target strand to remain intact after crosslinking, thus enabling sequence-specified detection of RNA, as well as DNA, targets. Following the methods described herein, hybridization and crosslinking of the side chains is inhibited if both probes are not bound simultaneously to the DNA or RNA target.

In another aspect, illustrated in FIG. 2, a three-probe set is provided having first, second, and third targeting sequences substantially complementary to substantially contiguous portions of a target sequence, with the probes comprising the first and third targeting sequences flanking the central probe comprising the second targeting sequence on the 3′ and 5′ sides. In a preferred embodiment, the central probe comprising the second targeting sequence serves as a capture probe incorporating the detection position, whereas the flanking probes comprising the first and third targeting sequences together comprise at least one crosslinking agent.

The three-probe system described herein is advantageous in diminishing the constraints imposed upon capture probe design. In the probe systems described in the prior art, in which the probes crosslinked to the target only, the capture probes had to be designed not only to exhibit target discrimination but also to permit crosslinking to the target. The second of these constraints is effectively removed from consideration in the present system, thereby simplifying the task of capture probe design. Thus, with this embodiment, the need for a crosslinking site (typically thymine) near the SNP site in the target sequence is alleviated, and, in addition, more sites for introducing detectable labels are provided as part of the probe set.

In one embodiment, the probes contain one or more photoactive coumarin-based crosslinking agents, such as those described in U.S. Pat. No. 6,005,093, the disclosure of which is incorporated by reference herein. In an alternative embodiment, these probes contain a photoactive aryl-olefin crosslinking agent as described in U.S. Pat. No. 6,303,799, the disclosure of which is incorporated by reference herein. As a further advantage of the instant invention, the probe pairs and probe sets provided herein permit crosslinking in the stem to include reactions between pairs of unnatural nucleotide analogs, thereby expanding the available options from which to select an appropriate choice of reactants.

Generally, the methods of the present invention comprise combining a probe pair or a probe set as described above with a sample comprising a target sequence, which may be present as a major component of the DNA from the target or as one member of a complex mixture. In a preferred embodiment, the method comprises the detection of a SNP of interest, e.g., a genotyping reaction. An interrogation region having a position for which sequence information is desired, generally referred to herein as the “interrogation position,” may be detected using probe sets complementary to portions of the interrogation region as described herein.

In one such embodiment, the interrogation position is a single nucleotide, although in some embodiments, it may comprise a plurality of nucleotides, either contiguous or separated by one or more nucleotides within the interrogation region. As used herein, the corresponding probe base that hybridizes with the interrogation position base in a hybridization complex is termed the “detection position.” In the case where the detection position is a single nucleotide, the nucleotide in the probe that has perfect complementarity to the detection position is called a “detection nucleotide.” Probes designed to hybridize with at least a portion of the interrogation region in a target sequence are generally referred to herein as “detection probes,” whereas the subset of such probes comprising a detection position is referred to herein as “allele-specific detection probes.”

Following the methods of the present invention, increased target detection sensitivity and improved data reliability can be obtained. Use of crosslinkable probes directed to one or more SNPs and/or other polymorphisms, such as gene deletion and duplication abnormalities, allows for high-stringency washes of the hybridized probe-target complexes, which significantly lower background contamination levels and result in improvements in the signal-to-noise ratio. Additional probe pairs or probe sets directed towards other polymorphisms in the gene or genes of interest may also be employed concurrently in the same platform for the same clinical sample, thereby providing a more complete genetic profile of a given locus or loci in parallel with one or more additional genotype determinations. For example, methods and compositions for determining gene dosage in isolation and in parallel with other genetic polymorphisms is described in U.S. patent application Ser. No. 10/093,626, the disclosure of which is incorporated by reference herein.

As will be appreciated by those in the art, the sample may comprise any number of things, including, but not limited to, bodily fluids (including, but not limited to, blood, urine, serum, lymph, saliva, anal and vaginal secretions, perspiration, and semen) or solid tissue samples of virtually any organism, with mammalian samples being preferred and human samples being particularly preferred; environmental samples (including, but not limited to, air, agricultural, water and soil samples); biological warfare agent samples; research samples; purified samples, such as purified genomic DNA, RNA, etc.; raw samples, such as bacteria, virus, genomic DNA, mRNA, etc. As will be appreciated by those in the art, virtually any experimental manipulation may have been done on the sample.

As used herein, the term “nucleotide” includes nucleotides as well as nucleoside and nucleotide analogs, and modified nucleotides such as labeled nucleotides. In addition, “nucleotide” includes non-naturally occurring analog structures, such as those in which the sugar, phosphate, and/or base units are absent or replaced by other chemical structures. Thus, the term “nucleotide” encompasses individual peptide nucleic acid (PNA) (Nielsen et al., Bioconjug. Chem. 1994; 5(1):3-7) and locked nucleic acid (LNA) (Braasch and Corey, Chem. Biol. 2001; 8(1):1-7) units. Similarly, the term “nucleotide” includes both individual units of ribonucleic acid (RNA) and deoxyribonucleic acid (DNA).

By “nucleic acid” or “oligonucleotide” or grammatical equivalents herein means at least two nucleotides covalently linked together. As will be appreciated by those of skill in the art, various modifications of the sugar-phosphate backbone may be done to facilitate the addition of labels or to increase the stability of such molecules in physiological environments. The nucleic acid may be single-stranded or double-stranded, as specified, or contain portions of both double-stranded and single-stranded sequences. The nucleic acid may be composed of DNA, both genomic and cDNA, RNA, or a hybrid, where the nucleic acid contains any combination of deoxyribo- and ribo-nucleotides, and any combination of bases, including uracil (U), adenine (A), thymine (T), cytosine (C), guanine (G), inosine, xathanine hypoxathanine, isocytosine, isoguanine, etc.

The compositions and methods of the invention are directed to the detection, quantification, and/or genotyping of target sequences. The term “target sequence” or “target nucleic acid” or grammatical equivalents herein means a nucleic acid sequence on a single strand of nucleic acid. In a preferred embodiment, the target sequence comprises a SNP. In another embodiment, the target sequence further comprises an additional polymorphism of interest, e.g., a dosage region. Alternatively, the sample may comprise a plurality of distinct target sequences, each having one or more polymorphisms of interest. By “plurality” as used herein is meant at least two.

The target nucleic acid may come from any source, either prokaryotic or eukaryotic, usually eukaryotic. The source may be mammalian, bacterial, or viral, wherein the genetic material (e.g., host genome, plasmid DNA, a PCR amplification product, or the like) may be naturally occurring or serving as a vector for DNA from a different source. The target DNA may be a particular allele of a mammalian host, an MHC allele, a sequence coding for an enzyme isoform, a particular gene or strain of a unicellular organism, or the like. The target sequence may be a portion of a gene, a regulatory sequence, genomic DNA, cDNA, RNA including mRNA and rRNA, or others. As is outlined herein, the target sequence may be a target sequence from a sample, or a secondary target such as a product of a genotyping or amplification reaction such as a ligated circularized probe, an amplicon from an amplification reaction such as PCR, etc. Thus, for example, a target sequence from a sample may be amplified to produce a secondary target (amplicon) that is detected. Alternatively, what may be amplified is the probe sequence, although this is not generally preferred. Therefore, as will be appreciated by those in the art, the target sequence may take many forms. For example, it may be contained within a larger nucleic acid sequence, i.e., all or part of a gene or mRNA, a restriction fragment of a cloning vector or genomic DNA, among others. As is outlined more fully below, probes are made to hybridize to a target sequence in order to determine the presence, sequence, or quantity of a target sequence in a sample. Generally speaking, the term “target sequence” will be understood by those skilled in the art.

If required, the target sequence is prepared using known techniques. For example, the sample may be treated to free the genetic material contained within via cellular lysis using known lysis buffer compositions, sonication, electroporation, etc., with subsequent purification and amplification steps performed as needed, as will be appreciated by those in the art. The sample may be a cellular lysate, isolated episomal element, e.g., YAC, plasmid, etc., virus, purified chromosomal fragments, cDNA generated by reverse transcriptase, amplification product, mRNA, etc. Depending on the source, the nucleic acid may be freed of cellular debris, proteins, DNA (if RNA is of interest), RNA (if DNA is of interest), size selected, gel electrophoresed, restriction enzyme digested, sheared, fragmented by alkaline hydrolysis, or the like. Importantly, however, and unlike the prior art, the benefits of improved sensitivity and reproducibility may be obtained following the methods of the present invention even without such additional sample purification steps.

The target sequence may be of any length, with the understanding that longer sequences are more specific. In one embodiment, the target nucleic acid is provided with an average size in the range of about 0.25 to 3 kilobases (kb). Nucleic acids of the desired length can be achieved, particularly with DNA, by restriction enzyme digestion, use of PCR and primers, boiling of high molecular weight DNA for a prescribed time, and the like. Desirably, at least about 80 mol %, usually at least about 90 mol % of the target sequence, will have the same size. For restriction enzyme digestion, a frequently cutting enzyme may be employed, usually an enzyme with a four-base recognition sequence, or a combination of restriction enzymes may be employed, where the DNA will be subject to complete digestion.

Preferably, double-stranded target nucleic acids are denatured to render them single-stranded in order to permit hybridization of the probe pairs and probe sets of the present invention. A preferred embodiment utilizes a thermal step, which generally involves raising the temperature of the reaction to about 95° C. in an alkaline environment, although chemical denaturation techniques may also be used. Where chemical denaturation has occurred, the pH of the medium will normally be adjusted to permit hybridization by the probes. Various reagents can be employed for adjusting the pH, particularly using mild acid solutions and buffers, such as those composed of acetic acid, citric acid, etc. The particular composition of the buffer employed is selected such that hybridization can occur during the subsequent incubation with the probes while affording the opportunity to apply the desired stringency condition.

The reactions outlined herein may be accomplished in a variety of ways, as will be appreciated by those in the art. Components of the reaction may be added simultaneously, or sequentially, in any order, with preferred embodiments outlined below. In addition, the reaction may include a variety of other reagents that may be included in an assay. These reagents include salts, buffers, innocuous proteins (e.g., albumin), detergents, etc., that may be used to facilitate optimal hybridization and detection, and/or reduce non-specific interactions. Also, reagents that otherwise improve the efficacy of the assay, such as protease inhibitors, nuclease inhibitors, anti-microbial agents, etc., may be used, depending on the sample preparation methods and purity of the target.

The term “allele” refers to individual genes that occupy the same locus or position on homologous chromosomes. When a subject has two identical alleles of a gene, the subject is said to be “homozygous” for the gene. When a subject has two different alleles of a gene, the subject is said to be “heterozygous” for the gene. Alleles of a specific gene can differ from each other in a single nucleotide or several nucleotides, and can include substitutions, deletions, and insertions of nucleotides. An allele of a gene can also be a form of a gene containing a mutation. A given allele can therefore be defined by a multitude of sequence variations, which are referred to herein as “allelic variants.”

“Mutation” is a relative term meant to indicate a difference in the identity of a base at a particular position, termed the “interrogation position” herein, between two or more sequences. In general, sequences that differ from the norm (referred to herein by the term “wild type”) are herein referred to by the term “mutant.” However, particularly in the case of SNPs, what sequence represents wild type may be difficult to determine as multiple alleles can be observed relatively frequently in the population, and thus what constitutes a mutant in this context requires the artificial adoption of one sequence as a standard (i.e., wild type).

The present invention provides probes that hybridize to adjacent or substantially contiguous portions of a target sequence, wherein the portions of the target sequence together define one or more regions of interest. Such adjacent or contiguous portions may or may not be separated by intervening nucleotides. Preferably, such adjacent or contiguous portions will be separated by less than five intervening nucleotides, more preferably less than three, still more preferably less than two, and most preferably the portions will be perfectly contiguous with each other. Thus, by “adjacent” or “substantially contiguous” as used herein is meant portions of a continuous nucleic acid sequence that are separated by no more than four nucleotides.

In general, the probes of the present invention are designed to be complementary to the interrogation region of the target sequence or to portions of other probes, such that hybridization may occur between a probe and its target and/or another probe. This complementarity need not be perfect; there may be any number of base-pair mismatches that will interfere with hybridization between a probe sequence (e.g., detection region) and its corresponding target sequence or another probe. However, if the degree of non-complementarity is so great that hybridization between a probe and its target cannot occur under even the least stringent of conditions, the probe sequence is considered to be not complementary to the target sequence. Thus, by “substantially complementary” herein is meant a situation where a probe sequence is sufficiently complementary to the corresponding region of its target sequence (e.g., interrogation region) and/or another probe to hybridize under the selected reaction conditions.

Hybridization generally depends on the ability of denatured DNA to anneal to nucleic acid strands bearing substantially complementary sequences in an environment below the melting temperature of the hybridized adduct. The higher the degree of complementarity between the pair of DNA sequences, the higher the relative temperature that can be used while maintaining the potential for hybridization. As a result, it follows that employing higher relative temperatures would tend to make the reaction conditions more stringent, whereas lower temperatures less so. For additional details concerning stringency of hybridization reactions, see Current Protocols in Molecular Biology, Ausubel et al. (Eds.).

Generally, the length of a probe and its GC content will determine the thermal melting point (Tm) of a hybridization complex, and thus contribute to defining the hybridization conditions necessary for obtaining specific hybridization of a probe to a region of interest. These factors are well known to a person of skill in the art, and can be tested experimentally. The Tm is the temperature (under defined ionic strength and pH) at which 50% of the hybridization complex exists in solution. An extensive guide to hybridization of nucleic acids is found in Tijssen, Hybridization with Nucleic Acid Probes: Theory and Nucleic Acid Probes, Vol. 1, 1993. Stringent conditions are generally defined to be about 5° C. lower than the Tm for a specific sequence at a defined ionic strength and pH. Highly stringent conditions are generally defined to be greater than or equal to the Tm. Sometimes the term “dissociation temperature” (“Td”) is used to define the temperature at which half of the probe is dissociated from its complementary sequence. In any case, a variety of techniques for estimating the Tm or Td are available, and described generally in Tijssen, supra. Typically, G-C base pairs in a duplex are estimated to contribute about 3° C. to the Tm, whereas A-T base pairs are estimated to contribute about 2° C., up to a theoretical combined maximum of about 80-100° C. However, more sophisticated models for Tm and Td determinations are available, and their application may be more appropriate if G-C stacking interactions, solvent effects, and the like need to be considered. For example, probes can be designed to have a desired dissociation temperature by using the formula: Td=(((((3×#GC)+(2×#AT))×37)−562)/#bp)−5; where #GC, #AT, and #bp are the number of G-C base pairs, the number of A-T base pairs, and the number of total base pairs, respectively, involved in the hybridization complex.

The stability difference between a perfectly matched duplex and a mismatched duplex, particularly if the mismatch is only a single base, can be quite small, corresponding to a difference in melting temperatures of as little as 0.5° C. Tibanyenda et al., Eur. J. Biochem. 1984; 139(1):19-27 and Ebel et al., Biochemistry 1992; 31(48):12083-12086. More importantly, it is understood that as the length of the region of complementarity increases, the effect of a single-base mismatch on overall duplex stability decreases. Thus, where there is a likelihood of unknown mismatches between the probe sequence and the target sequence, it may be advisable to use a longer complementary region in the probe, if the formation of a hybridized duplex is desired. Alternatively, if one wishes to probe a polymorphic interrogation position with a plurality of allele-specific detection (e.g., capture) probe, it may be advisable to use a shorter complementary region in the probe in order to improve discrimination among the different allele sequences.

Thus, the specificity and selectivity of a probe can be adjusted by choosing the proper length for the region of complementarity in conjunction with appropriate hybridization conditions. When the sample is genomic DNA (e.g., mammalian genomic DNA) the selectivity of the probe sequences must be high enough to identify the correct sequence in order to allow processing directly from genomic DNA. However, in situations in which a portion of the genomic DNA is first isolated from the rest of the DNA, e.g., by separating one or more chromosomes from the rest of the chromosomes, a probe with a more relaxed set of design requirements may be sufficient to achieve the desired selectivity or specificity.

The appropriate length for a probe, and therefore the hybridization conditions, will also depend on whether a single probe is hybridized to a target sequence, or several probes. In a preferred embodiment, several probes are used and all of them must be able to hybridize to the target sequence simultaneously. With this embodiment, it is desirable to design the probes such that their Tm or Td values are similar such that all of the probes may hybridize specifically to the target sequence. These conditions can be determined by a person of skill in the art by taking into consideration the factors discussed above.

A variety of hybridization conditions may be used in the present invention, including high-, moderate-, and low-stringency conditions; see, e.g., Sambrook et al., Molecular Cloning: A Laboratory Manual, 2nd ed., 1989 and Short Protocols in Molecular Biology, Ausubel et al. (Eds.), 1992, hereby incorporated by reference. Stringent conditions are sequence-dependent and will differ depending on specific circumstances. Longer sequences hybridize more specifically at higher temperatures. Stringent conditions will be those in which the salt (e.g., sodium chloride) concentration is less than about 1.0 M, typically between 0.01 to 1.0 M at pH 7.0 to 8.3, and the temperature is at least about 30° C. for short probes (e.g., 10 to 50 nucleotides (nt)) and at least about 60° C. for long probes (e.g., greater than 50 nt) in an entirely aqueous hybridization medium. Stringent conditions may also be achieved with the addition of helix destabilizing agents such as formamide. The hybridization conditions may also vary when a non-ionic probe backbone (e.g., PNA) is used, as is known in the art.

Thus, the assays are generally run under stringency conditions that allow formation of the hybridization complex only in the presence of the target. Stringency can be controlled by altering a parameter that is a thermodynamic variable, including, but not limited to, temperature, salt concentration, chaotrope concentration, pH, organic solvent (e.g., formamide) concentration, etc. These parameters may also be used to control non-specific binding, as is generally outlined in U.S. Pat. No. 5,681,697. Thus it may be desirable to perform certain steps at higher stringency conditions to reduce non-specific binding, as described herein. The skilled artisan will recognize how to adjust the temperature, ionic strength, etc. as necessary to accommodate factors such as probe length and the like.

The probes that bind to the target will generally be composed of naturally occurring nucleotides, but in some may contain one or more nucleotides bearing unnatural features such as a modified or an absence of a sugar unit; a modified sugar-phosphate chain where one or more oxygen atoms of the phosphate are substituted by sulfur, carbon, and/or nitrogen atoms, or the like; a modified or an absence of a base unit; or other modification that can provide synthetic advantages, stability under the conditions of the assay, resistance to enzymatic degradation, etc. In one embodiment, modified nucleotides that do not significantly impact the Tm values are incorporated into the probes.

As will be appreciated by those in the art, the probes of the invention may take on a variety of configurations. The desired probe will have a targeting sequence of at least about 10 nt, more usually at least about 15 nt, preferably at least about 16 or 17 nt and usually not more than about 1 kb, more usually not more than about 0.5 kb, preferably in the range of about 18 to 200 nt, and frequently not more than 50 nt, where the targeting sequence is substantially complementary to the corresponding portion of the target sequence, as described herein.

In the preferred embodiment, one or more allele-specific detection probes are provided having targeting sequences substantially complementary to the interrogation region upstream and downstream of an interrogation position for which sequence information is desired, but differing in the corresponding interrogation nucleotides. In this embodiment, the targeting sequences are substantially complementary to the sequence surrounding the polymorphism at the interrogation position, but differ at the corresponding interrogation position with respect to the wild-type and mutant sequences, thereby enabling discrimination between normal and mutant genotypes, as described herein.

As a central aspect of the present invention, the probes that make up a probe pair or set may further comprise one or more side chains. In the embodiment comprising a probe pair, the side chains are located on the 3′ end of one probe and the 5′ end of the other probe. In the three probe set, an additional probe having side chains located on both the 3′ and 5′ ends of the targeting sequence is provided positioned between a flanking probe pair. Upon hybridization of the targeting sequences of the probes to their respective portions on the target sequence, the side chains provide for non-covalent association to form stem structures. Non-covalent association can be obtained by hydrogen bonding, salt bridges, van der Waal's forces, and the like, particularly hydrogen bonding. For the most part, the groups involved in hydrogen bonding will be composed of oxygen- and nitrogen-containing donor/acceptor units, e.g., purines and pyrimidines. Upon activation, covalent crosslinking between the side chains comprising a stem occurs. Owing to the spatial proximity of the side chains due to stem formation, the reaction rate of crosslinking will usually be at least about 10-fold, preferably at least about 100-fold, greater than that which would occur in the absence of stem formation.

The side chains are designed to have a weak association or affinity to other sequences in the reaction mixture. By weak is intended that in the absence of the target in the solution, the equilibrium between unassociated probes and associated probes (thereby forming a stem) will greatly favor the dissociated state, where the association constant is less than about 10⁻¹, usually less than about 10⁻³ M⁻¹. The affinity may result from hydrogen bonding, salt formation, and/or other relatively weak chemical interaction(s).

To obtain stem formation, one may use paired nucleotides, at least 2 base pairs, generally at least 3 base pairs, and usually not more than about 20 base pairs, more usually not more than about 16 base pairs, preferably not more than about 8 base pairs, more preferably not more than about 6 base pairs, usually in the range of 2 to 6 base pairs, more usually in the range of 4 to 6 base pairs. Alternatively, one may use amino acids which provide for hydrogen bonding and/or salt bridges. Other groups that may form hydrogen bonds include diamines and diol functionalities, particularly the ones found in ortho-phenolates. However, for the most part, considering convenience, ease of synthesis, control of affinity, and substantial absence of interference, nucleotides, nucleotide analogs, or nucleotide derivatives where, for example, the sugar or phosphate groups may be substituted, or the amino or oxo groups of the base may be modified, or the like, will be employed. Usually, the base pairs will involve A and T, where the nucleotides may be the same on one side chain or different; that is, one side chain may be composed of all T nucleotides whereas the other composed of all A nucleotides, or each side chain may be composed of a mixture of A and T nucleotides. However, one may also use G and/or C, by themselves or in combination with A and T. Instead of the normal 4 or 5 natural bases (including uracil), one may use other bases or other moieties providing for hydrogen bonding and spatial orientation, such as 5-methylcytosine, 5-fluorouracil, 2′-deoxy-5-(trifluoromethyl)uridine, inosine, 1-methylinosine, 3-nitropyrrole, and the like. The particular choice of nucleotide or substitute moiety will depend on the desired affinity, ease of synthesis, interaction with the covalent crosslinking moiety, opportunity to serve as a reactant for crosslinking, and the like.

Generally, the side chain backbone, which excludes groups bound to the chain, will be at least about 15 atoms in length, more usually at least about 20 atoms, generally fewer than 100 atoms, and more usually fewer than about 60 atoms. The atoms making up the backbone will be carbon, oxygen, nitrogen, sulfur, phosphorus, or the like. The crosslinking moiety may be part of the side chain or appended to the side chain, depending upon the nature of the moiety.

In addition, a probe may further comprise one or more labels (including ligands), such as a radiolabel, fluorophore, chemilumiphore, fluorogenic substrate, chemilumigenic substrate, biotin, antigen, enzyme, photocatalyst, redox catalyst, electroactive moiety, a member of a specific binding pair, or the like, that allow for capture or detection of a crosslinked probe. The label(s) may be bonded to any convenient location of the probe chain so long as it does not interfere significantly with its ability to hybridize with the target sequence. Labels will generally be small, usually from about 100 to 1,000 Da. The labels may be any detectable entity, where the label is detected directly or indirectly by being recognized by a receptor (e.g., an antibody), which in turn is labeled with a molecule that is readily detectable. Molecules that provide for detection in electrophoresis include radiolabels (e.g., ³²P, ³⁵S, etc.), fluorescers (e.g., rhodamine, fluorescein, etc.), ligands for receptors and antibodies (e.g., biotin for streptavidin, digoxigenin for anti-digoxigenin, etc.), chemiluminescers, and the like. Alternatively, the label may be capable of providing a covalent attachment to a solid support such as a bead, plate, slide, or column of glass, ceramic, or plastic.

As indicated herein, the methods of the present invention utilize probe pairs and probe sets capable of crosslinking to a target sequence and to each other via the stem(s) formed by the side chains. There are extensive methodologies for achieving covalent crosslinking upon hybridization between a probe and a target, or between associated side chains. Conditions for activation may include photonic, thermal, and chemical methodologies. Although the primary method is photonic, it may be used in combination with other methods of activation. Therefore, photonic activation will be primarily discussed as the method of choice, but for completeness, alternative methods will be mentioned briefly.

A given probe will have from 0 to 5 crosslinking agents, more usually from about 1 to 3 crosslinking agents. The crosslinking agents must be capable of forming a covalent crosslink between the probe and target sequence, and will be selected so as not to interfere with hybridization. In a preferred embodiment, the crosslinking agents will in a hybridized complex be positioned across from T, C, or U bases in the target sequence. As indicated above, a significant advantage of the compositions and methods described herein is the ability to detect polymorphic sequences of interest lacking a suitable, naturally-occurring base in close proximity to the polymorphic region to be detected. A design capable of delivering this advantage is provided by the crosslinkable three probe set illustrated in FIG. 2, which in addition has been shown in an experiment (see Example 2) to provide about a three-fold improvement in signal-to-noise compared with the probe set design applied in the prior art.

For the most part, the compounds employed for crosslinking will be photoactivatable compounds that can form covalent bonds with a base, particularly a pyrimidine. These compounds will include functional moieties, such as coumarin, as present in substituted coumarins, furocoumarin, isocoumarin, bis-coumarin, psoralen, etc.; quinones, pyrones, α,β-unsaturated acids; acid derivatives (e.g., esters); ketones; nitriles; azido compounds; etc. A large number of functionalities can be generated photochemically and can form a covalent bond with almost any organic moiety. These groups include carbenes, nitrenes, ketenes, free radicals, etc. One can provide for a scavenging molecule in the bulk solution, normally excess non-target nucleic acid, so that probes that are not bound to a target sequence will react with the scavenging molecules to avoid non-specific crosslinking between probes and target sequences. Carbenes can be obtained from diazo compounds, such as diazonium salts, sulfonylhydrazone salts, or diaziranes. Ketenes are available from diazoketones or quinone diazides. Nitrenes are available from aryl azides, acyl azides, and azido compounds. For further information concerning photolytic generation of an unshared pair of electrons, see Schoenberg, Preparative Organic Photochemistry, 1968.

Another class of photoactive reactants are inorganic/organometallic compounds based on any of the d- or f-block transition metals. Photoexcitation induces the loss of a ligand from the metal, thereby providing a vacant site for substitution. Suitable ligands include nucleotides. For further information regarding the photosubstitution of metal complexes, see Geoffrey and Wrighton, Organometallic Photochemistry, 1979.

In one preferred embodiment, the crosslinking agent comprises a coumarin derivative as described in co-pending U.S. patent application Ser. No. 09/390,124 and in U.S. Pat. No. 6,005,093, the disclosures of which are incorporated herein in their entirety. Briefly, with this embodiment the probes of the present invention benefit from having one or more photoactive coumarin derivatives attached to a stable, flexible, (poly)hydroxy hydrocarbon backbone unit. Suitable coumarin derivatives are derived from molecules having the basic coumarin ring system, such as the following: 1) coumarin and its simple derivatives; 2) psoralen and its derivatives, such as 8-methoxypsoralen or 5-methoxypsoralen (at least 40 other naturally occurring psoralens have been described in the literature and are useful in practicing the present invention); 3) cis-benzodipyrone and its derivatives; 4) trans-benzodipyrone and its derivatives; and 5) compounds containing fused coumarin-cinnoline ring systems. All of these molecules contain the necessary crosslinking group (i.e., an activated double bond) to crosslink with a nucleotide in a hybridized nucleic acid strand.

Another preferred embodiment utilizes aryl-olefin derivatives as the crosslinking agent, as described in U.S. patent application Ser. No. 09/189,294 and corresponding U.S. Pat. No. 6,303,799, the disclosures of which are incorporated herein in their entirety. In this embodiment, the aryl-olefin unit contains a photoactivatable double bond that can covalently crosslink to suitable reactants in the complementary strand. Thus, the aryl-olefin unit can serve as a crosslinking moiety when attached via a linker to a suitable backbone moiety incorporated into the probe sequence.

The probes may be prepared by any convenient method, most conveniently synthetic procedures, where the crosslinker-modified nucleotide is introduced at the appropriate position stepwise during the synthesis. Alternatively, the crosslinking molecules may be introduced into the probe through photochemical or chemical monoaddition. The above patent disclosures provide specific teachings regarding the incorporation of coumarin and aryl-olefin derivatives, which are incorporated by reference herein. Linking of various molecules to nucleotides is well known and fully described in the literature, and hence does not require elaboration here. See, for example, Oligonucleotides and Analogues: A Practical Approach, Echstein (Ed.), 1991.

The probes and target will be brought together in an appropriate medium under conditions that allow the desired stringency to be applied in the assay. Therefore, usually buffered solutions will be used, employing reagents, such as sodium citrate, sodium chloride, Tris, EDTA, EGTA, magnesium chloride, etc. See, for example, Sambrook et al., Molecular Cloning: A Laboratory Manual, 1988, for a list of various buffers and conditions, which is not an exhaustive list. Solvents may include water, formamide, DMF, DMSO, HMP, alkanols, and the like, individually or in combination, usually aqueous-based solvents. Temperatures may range from ambient to elevated temperatures, usually not exceeding about 100° C., more usually not exceeding about 90° C. Usually, the temperature for photochemical and chemical crosslinking will be in the range of about 20 to 70° C. For thermal crosslinking, the temperature will usually be in the range of about 70 to 120° C.

The amount of target nucleic acid in the assay medium will generally range from about 0.1 yoctomole to about 100 picomoles, more usually 1 yoctomole to 10 picomoles. The concentration of sample nucleic acid will vary widely depending on the nature of the sample. Concentrations of sample nucleic acid may vary from about 0.01 femtomolar to 1 micromolar. Similarly, the ratio of probe to target nucleic acid in the assay medium may vary, or be varied widely, depending on the amount of target in the sample, the number and types of probes included in the probe mixture, the nature of the crosslinking agent, the detection methodology, the length of the complementarity region(s) between the probe(s) and the target, the differences in the nucleotides between the target and the probe(s), the proportion of the target nucleic acid to total nucleic acid, the desired amount of signal amplification, or the like. Each probe may be about at least equimolar to the target concentration but is usually in substantial excess. Generally, each probe will be in at least 10-fold excess, and may be in 10⁶-fold excess, usually not more than about 10¹²-fold excess, more usually not more than about 10⁹-fold excess in relation to the target concentration. The ratio of one probe (e.g., capture probe) to another probe (e.g., a reporter probe) in the overall probe mixture may also vary based on considerations discussed above.

Conveniently the stringent washes will employ buffers composed of about 1× to 10× saline-sodium citrate (SSC) or similar composition. The solution may also contain a small amount of an innocuous protein (e.g., serum albumin, beta-globulin, etc.) generally added to a concentration in the range of about 0.5 to 2.5%. DNA hybridization may occur at an elevated temperature, generally ranging from about 20 to 70° C., more usually from about 25 to 60° C. The incubation time may be varied widely, depending on the nature of the sample, generally being at least about 5 minutes and not more than 6 hours, more usually at least about 10 minutes and not more than 2 hours.

After sufficient time for hybridization has elapsed, the crosslinking agent may be activated to provide crosslinking. The activation may involve illumination, heat, chemical reagent, or the like, and will occur through actuation of an activator, e.g., a means for introducing a chemical agent into the medium, a means for modulating the temperature of the medium, a means for irradiating the medium, and the like. If the activatable group is a photoactivatable group, the activator will be an irradiation means where the particular wavelength that is employed may vary from about 250 to 650 nm, more usually from about 300 to 450 nm. The illumination power will depend upon the particular reaction and may vary in the range of about 0.5 to 250 W. Activation may then be initiated immediately or after a short incubation period, usually less than 1 hour, more usually less than 0.5 hour. With photoactivation, usually extended periods of time will be involved with the activation, where incubation occurs concurrently. The photoactivation time will usually be at least about 1 minute and not more than about 2 hours, more usually at least about 5 minutes and not more than about 1 hour.

The purpose of introducing covalent crosslinks between the probes and target nucleic acid, and between the probes themselves, is to raise effectively the Tm of the complex above that attained by hydrogen bonding alone. This property allows wash steps to be performed at greater stringency than under the initial hybridization conditions, thereby allowing marked reduction in the amount of non-specifically bound adducts. Thus, the methods of the present invention provide for the formation of hybridized complexes in which the probes are linked to their respective target sequences and/or to each other not just through hydrogen bonding but also covalent bonding. Therefore, harsher conditions that will disrupt any undesirable, non-specific background binding, but at the same time not break the covalent bond(s) linking the probe to its crosslinked partner(s), may be employed. For example, washes with urea-containing solutions or alkaline solutions could be used. Heat could also be used. The covalent linkage therefore allows for a significant improvement in the signal-to-noise ratio of the assay.

As described above, high-stringency conditions for the wash step generally employ conditions of low ionic strength and high temperature, or alternatively a denaturing agent, such as formamide. In a preferred embodiment, the wash conditions are 1×SSC, 0.1% Tween® 20 at room temperature (20-25° C.). In another preferred embodiment, the wash conditions are 50% formamide, 0.5% Tween® 20, 0.1×SSC at room temperature (20-25° C.).

After crosslinking of the hybridized probes to the target sequence and/or to each other, the label(s) incorporated into the probe(s) may be detected. A number of different labels that can be used with the crosslinkable probes are known in the art. For example, using a label that is a member of a specific binding pair (e.g., antigen and antibody, such as digoxigenin and anti-digoxigenin; biotin and streptavidin; sugars and lectins; etc.) allows one to isolate the crosslinked nucleic acid products onto a solid support (e.g., container surface, magnetic bead, or the like). Having a label that may provide a detectable signal, either directly or indirectly, where the label can be crosslinked to the target nucleic acid, affords the opportunity to detect when said crosslinked nucleic acid has been separated onto a solid support or in some manner isolated. For direct detection, the label may be a fluorophore, chemiluminescer, radiolabel, or the like. For indirect detection, one will usually have a ligand that binds to a reciprocal member, which in turn is labeled with a detectable label. The label may be any of the above labels, as well as an enzyme, where one can determine the presence of crosslinked probe by adding an enzyme substrate. Alternatively, this label may be a member of binding pair whose reciprocal partner generates a detectable signal, such as through the action of an enzyme on a substrate.

In one embodiment, at least one probe of the probe pair or three probe set includes a label comprising a member of a specific binding pair (e.g., biotin) in order to achieve separation of the DNA of interest from the remainder of the sample. In a preferred embodiment, at least one such label is included in the center capture probe comprising the second targeting sequence in the three probe set. In alternative embodiments, the capture label may be incorporated into one or both flanking probes of the three probe set, or one or both probes of the nucleic acid probe pair.

In a further preferred embodiment, at least one of the probes in the probe pair or probe set also comprises a reporter label providing a detectable signal. A quantitative measurement may then be obtained by comparing the signals observed from the sample and from a control. In a preferred embodiment, a plurality of reporter probes are employed that not only contain crosslinking nucleotides to allow photocrosslinking to the target, but also are polyfluoresceinated (or equivalently multifluoresceinated) to provide for increased signal generation. In a further specification of this preferred embodiment, the reporter probes bear no side chains and possess targeting sequences complementary to portions of the target sequence that do not overlap with those associated with the targeting sequences of any other probe.

A detectable signal may be generated by using a reagent such as AttoPhos® Substrate, as described herein, or other substrates that are transformed into fluorescent, chemiluminescent, or colored products in the presence of appropriate enzymes. With the present invention, the same sample can be contacted with different probe mixtures in different wells of the same microtiter plate in order to assay concurrently for gene dosage abnormalities such as deletions and duplications, as well as sequence differences such as SNPs. In an alternative embodiment, capture probes may be linked covalently to a solid support prior to performance of the assay.

Instead of separating the crosslinked probes-target DNA complex from the assay medium, detection techniques can also be employed that allow for detection during the course of the assay. Alternatively, gel electrophoresis may be employed, and the amount of crosslinked probe(s) to target determined by the presence of a radioactive label on the probe using autoradiography; by staining the nucleic acid and detecting the amount of dye that binds to the crosslinked probe; by employing an antibody that is specific for the crosslinked nucleic acid structures, particularly the crosslinked area, so that an immunoassay may be employed; or the like.

Multiple polymorphisms in one or more target sequences can be determined in parallel in accordance with the subject protocols. Clinical diagnostics is improved substantially with the present invention by the ability to assay simultaneously multiple mutational mechanisms of human genetic variation in a single platform, including both gene dosage and sequence abnormalities. The resulting genetic profile obtained for a given locus or loci will be more complete and, therefore, is better suited for risk profiling, chemopredictive testing, disease profiling, and pharmacogenetic testing, as well as for determining genetic mutations, genetic diseases, genotyping for trait analysis, and genotyping of other polymorphic sequences in humans, plants, and animals.

Specific genetic targets of interest include sequence variations such as SNPs. Generally, there may be a single nucleotide change in a single gene that is severe enough to cause disease in an individual (monogenic disease). However, there exist many other genetic sequence variations that do not necessarily cause a disease. These polymorphisms may, however, act in concert with one or several other genetic or environmental factors to produce a disease phenotype.

Kits comprising nucleic acid probe pairs or three probe sets capable of crosslinking, as described previously, are provided. The probes may be labeled or unlabeled to allow for easy isolation or detection of the crosslinked nucleic acids. One may use radioactive labels, fluorescent labels, specific binding-pair member labels, and the like. The probes include sequences for hybridizing and crosslinking to a target sequence, and side chains for hybridizing and crosslinking to each other via stem structures. In a preferred embodiment, the kit will comprise at least two probe sets directed to an interrogation region (e.g., SNP) of interest. As noted above, there may be a plurality of probe sets directed to additional target sequences to detect alternative polymorphisms that may be present in the gene or genes of interest. For example, pairs of probes may be used where the target sequence has a plurality of potential mutations spread throughout a gene. Ancillary materials may be provided, such as dyes, labeled antibodies, where a ligand is used as a label, and the like.

The following examples are offered by way of illustration and not by way of limitation.

### EXAMPLE 1

### Assay for Diagnosis of Factor V Leiden

Background

Factor V is a member in a set of proteins involved in blood coagulation. Factor V Leiden is a mutant variant that arises from a point mutation (G1691A) in the encoding gene. This mutation represents the most common inherited risk factor for thrombosis. An accurate, efficient, and inexpensive method for identifying the factor V genotype remains a desirable goal in clinical diagnostics. This example demonstrates the utility of the present invention for discriminating between and detecting for DNA targets that differ by only a single nucleotide.

Assay Design

The diagnostic assay described in this example utilizes three discrete types of oligonucleotide probes (capture, reporter, and flanking). FIG. 3 depicts schematically one possible arrangement of the probes and their interactions with the target and among themselves. As indicated in FIG. 3, (1) the capture probe does not crosslink to the target; (2) the flanking probes crosslink to both the target and the capture probe; (3) the number of potential crosslinking sites between each flanking or reporter probe and the target is at least one; (4) the number of flanking probes is at least one, but preferably two; and, (5) the number of reporter probes is at least one.

The novelty of the present invention for genetic mutation analysis is expressed in this example by a strategy that involves the creation of a crosslinked structure composed of the capture probe, two flanking probes, and the target DNA. A key feature of this assay design is that formation of this crosslinked structure is necessarily dependent on the simultaneous hybridization of the capture and flanking probes to the target.

Probe Design and Synthesis

The three-probe set in this example was designed to be complementary to exonic and intronic regions of the factor V gene within the context of the human genome. In the following oligonucleotide sequences, ‘X’ denotes the XL10 crosslinker nucleotide, whose description and synthesis is disclosed in co-pending U.S. Patent Application No. 60/408,633.

Allele-Specific Capture Probes

The allele-specific capture probes serve to provide a high degree of hybridization-based discrimination between targets harboring the wild-type and mutant sequences. The following two probes were designed to hybridize to either the wild-type factor V or the mutant factor V Leiden gene sequence:

| | |
|---|---|
| Capture Probe FV-1 | |
| 3′-biotin-AXAATAAGGAGCGGACAGTTT-5′ | (SEQ ID NO: 1) |
| Capture Probe FV-2 | |
| 3′-biotin-AXAATAAGGAACGGACAGGTTT-5′ | (SEQ ID NO: 2) |

Capture Probe FV-1 and Capture Probe FV-2 target the wild-type and mutant sequences, respectively. Each capture probe is biotinylated at the 3′ end, and the biotin-containing nucleotide is separated from the 3′ adenine by ten spacer nucleotide units. In addition, each capture probe bears a 3′ AXA and a 5′ TTT, which are used to form the stem structures and photocrosslink with the flanking probes.

Flanking Probes

The flanking probes serve to form duplex DNA stem structures with the ends of a captures probe. It is important to note that formation of the stem structures is conditional to hybridization of the capture probe to the target. The following three probes were designed to hybridize and photocrosslink to the target, and to form stem structures and photocrosslink with the ends of a hybridized capture probe:

| | |
|---|---|
| Flanking Probe FV-1 | |
| 3′-AXACTTTCCAATGAXGTTCCTGTTTTATGGACT | (SEQ ID NO: 3) |

TT-5′

| | |
|---|---|
| Flanking Probe FV-2 | |
| 3′-AXAGTCCCTAGACGAXAATGTCTAATCTTCATC | (SEQ ID NO: 4) |

AGGATXA-5′

| | |
|---|---|
| Flanking Probe FV-3 | |
| 3′-AXATCCCTAGACGAXAATGTCTAATCTTCATCA | (SEQ ID NO: 5) |

GGATXA-5′

Flanking Probe FV-1 and Flanking Probe FV-2 are used to sandwich Capture Probe FV-1, whereas Flanking Probe FV-1 and Flanking Probe FV-3 are used to sandwich Capture Probe FV-2. The 5′ TTT of Flanking Probe FV-1 is designed to form a stem structure and photocrosslink with the 3′ AXA of either capture probe. Meanwhile, the 3′ AXA of Flanking Probe FV-2 and Flanking Probe FV-3 is designed to form a stem structure and photocrosslink with the 5′ TTT of Capture Probe FV-1 and Capture Probe FV-2, respectively.

Multifluoresceinated Reporter Probes

The multifluoresceinated reporter probes serve to provide a detectable signal through an anti-fluorescein antibody-enzyme conjugate signal-generating system. The following seven probes were designed to hybridize and photocrosslink to the target:

| | |
|---|---|
| Reporter Probe FV-1 | |
| 3′-T-multifluorescein- | (SEQ ID NO: 6) |

TXATCCTTTCCGATTAGTTGAXC-5′
Reporter Probe FV-2

| | |
|---|---|
| 3′-T-multifluorescein- | (SEQ ID NO: 7) |

GGGACCAXCTTGACGAGACTAGTACCACAXC-5′
Reporter Probe FV-3

| | |
|---|---|
| 3′-T-multifluorescein- | (SEQ ID NO: 8) |

AXAGAGTGACTTGGGGGTTTGTCTGGAXA-5′
Reporter Probe FV-4

| | |
|---|---|
| 3′-T-multifluorescein- | (SEQ ID NO: 9) |

CTGGACCTTAXACTTTGATTCTGTTTTATACXA-5′
Reporter Probe FV-5

| | |
|---|---|
| 3′-T-multifluorescein- | (SEQ ID NO: 10) |

AXATCTGAACGGAXGCCGTCACTACCATGAC-5′
Reporter Probe FV-6

| | |
|---|---|
| 3′-T-multifluorescein- | (SEQ IDNO: 11) |

AXTAGTGTGACCACGATTTTTCCTGATGAXC-5′
Reporter Probe FV-7

| | |
|---|---|
| 3′-T-multifluorescein- | (SEQ ID NO: 12) |

AXTGACAAGAGAACTTCCTTTACGGGGTAATAXA-5′

Each reporter probe is fluoresceinated on the 3′ side of the oligonucleotide. Each fluorescein moiety is attached to a probe through a chemical reaction utilizing a functionalized nucleotide analog bearing one potential fluoresceination site. The functionalized nucleotide analogs are separated from one another by one spacer nucleotide unit; there are 40 repeating functionalized-spacer nucleotide pair units for a given reporter probe.

Synthesis

Oligonucleotides were synthesized using standard phosphoramidite chemistry on an automated platform. A more detailed procedure may be found in U.S. patent application Ser. No. 10/093,626. The protocol for fluoresceinating oligonucleotides to yield the multifluoresceinated reporter probes has been modified as follows:

1000 pmol of amine-functionalized oligonucleotide in 50 μL 100 mM NaHCO₃ (pH 8.0) were added to 50 μL of a DMSO solution containing 1.0 mg 5(6)-carboxyfluorescein-N-hydroxysuccinimide ester (FLUOS) (Roche). This reaction mixture was incubated overnight at room temperature. Unreacted FLUOS reagent was separated from the fluorescein-labeled probe by filtration using a Centricon® YM-30 (Millipore) centrifugal filter unit. Centrifugation was repeated 4 times with the addition of 2 mL of rinse solution (10 mM NaOH) prior to each spin. One final rinse spin was performed using 10 mM Tris-HCl (pH 7). Afterwards, the concentrated probe solution (˜30 μL) was diluted to 110 μL by the addition of 10 mM Tris-HCl (pH 7.5).

Sample Preparation

The samples used in the assay derived from human blood. Genomic DNA was extracted and purified from 200 μL volumes of blood using a QIAamp® Kit (QIAGEN). The yield of DNA typically ranged between ˜3-8 μg. The DNA was then digested with a restriction enzyme (Alu I) in order to fragment the DNA into shorter length pieces. The resulting material constituted the samples with which the crosslinking hybridization assays were performed.

Assay Procedure

Each sample was aliquoted into two wells (125 μL per well) of a 96-well polypropylene microtiter plate. To one aliquot was added Probe Mixture 1 (50 μL) containing Capture Probe FV-1, the flanking probes, and the reporter probes. To the other aliquot was added Probe Mixture 2 (50 μL) containing Capture Probe FV-2, the flanking probes, and the reporter probes. In addition to the samples, each assay plate included six negative controls (containing no target DNA) and two positive controls (DNA derived from a heterozygous individual). Probe Mixture 1 was added to three of the negative control wells and one of the positive control wells, and Probe Mixture 2 was added to the remaining three negative control wells and remaining positive control well. The negative controls were, therefore, run in triplicate for each capture probe.

Following the addition of the probe mixtures to the samples and controls, 50 μL neutralization reagent (190 mM citric acid, 300 mM NaH₂PO₄, 1.5 M NaCl, 4 mL/L Tween® 20, 350 mL/L formamide) was added to each well. The microtiter plate was then heated inside of a UV crosslinking chamber. The reaction mixtures were incubated for 20 min to 45° C. and then irradiated for 45 min at the same temperature. Afterwards, streptavidin-coated magnetic beads (Dynabeads® M-280 (Dynal)) were introduced into each well and the resulting mixture incubated for 30 min to allow binding of any species or adducts containing the biotin-conjugated capture probes. The beads were washed once with Wash I (1.5 mM NaCl, 0.15 mM sodium citrate, 0.1% SDS (pH 7)), once with Wash II (150 mM NaCl, 15 mM sodium citrate, 0.1% Tween® 20 (pH 7)), and then resuspended and incubated for 20 min in a solution (100 μL) containing a 1:20000 dilution of anti-fluorescein antibody-alkaline phosphatase conjugate (DAKO).

The beads were then washed four times with 225 μL Wash II. Upon completion of the final wash cycle, 100 μL of the alkaline phosphatase substrate AttoPhos® Substrate (Promega) was added to each well. The plate was incubated at 37° C. for 60 min. Fluorescence signal detection was achieved by using a FluoroCount™ fluorescence microplate reader (Packard).

Assay Results

Results obtained from a pool of 20 samples are presented below. The factor V genotype was determined by evaluating the ratio of the net sample signal (NSS) (i.e., background-subtracted) obtained from using Capture Probe FV-2 to the NSS obtained from using Capture Probe FV-1. In theory, a homozygous normal, heterozygous, and homozygous mutant sample will yield a NSS ratio of 0, 1, and ∞, respectively. The following table summarizes the experimentally derived data and their interpretation:

| Number of Samples | NSS Ratio Range | Interpreted Genotype |
|---|---|---|
| 16 | 0.18-0.37 | Homozygous normal |
| 2 | 1.28-1.41 | Heterozygous |
| 2* | 11.17-20.34 | Homozygous mutant |

*The same sample was used to run the assay twice.

Assay Evaluation

The results demonstrate that the crosslinking hybridization assay using the three-probe set strategy yields clearly interpretable data for factor V Leiden genotyping. The NSS ratios for the three possible genotypes fall within one of three distinct ranges. Importantly, the interpreted genotypes were found to be in 100% concordance with results obtained from subjecting each sample through an independent and established PCR-based test for factor V Leiden genotyping, thereby validating the clinical utility of this three-probe set assay.

### EXAMPLE 2

### Comparison of Assay Designs

Background

The effectiveness of crosslinking hybridization assays for gene mutation analysis has previously been described, for example, in U.S. Pat. No. 6,277,570 and U.S. patent application Ser. No. 10/093,626. The previous disclosures, however, described assay designs in which the capture probes crosslinked to the target and the probes did not crosslink among themselves.

One advantage of the current invention derives from the fact that, unlike the previously disclosed designs, the current approach does not require the capture probe to crosslink to the target. By eliminating this requirement from the capture probe design, the selection of a pair of discriminatory capture probes becomes a less burdensome task by obviating the need to consider not only the need to incorporate crosslinking sites between the capture probes and target, but also its effect on their target discrimination properties.

Another advantage of the current invention was demonstrated when the signal-generating properties of the current and previous designs were compared experimentally, as described below.

Experimental Design

Comparative tests were performed in which each test employed a different composition of capture and flanking probes. The capture probe used to assess the previous assay design had the following sequence:

| | |
|---|---|
| Capture Probe FV-3 | |
| 3′-biotin-AXAAGGAGCGGACAGGT-5′ | (SEQ ID NO: 13) |

The capture probe used to assess the current assay design was Capture Probe FV-1. The flanking probes were selected from among the ones listed in the previous example.

Each test used samples containing the same amount of target DNA, which were PCR amplicons (harboring the mutation site of the factor V gene) originating from genomic DNA of a heterozygous factor V Leiden individual. Each test used the seven factor V reporter probes listed above.

Procedure

The test assays were run in a fashion analogous to the assay procedure described in the previous example. Appropriate modifications were made to account for the different probe mixture compositions among the individual tests.

Results and Discussion

Results from a set of 5 tests are summarized in the following table:

| | | | | | Signal-to- |
|---|---|---|---|---|---|
| Test | Capture | Flanking | Signal | Background** | Background |
| Number | Probe | Probe(s) | (RFU*) | (RFU*) | Ratio |
| 1 | FV-3 | none | 2370 | 78 | 30 |
| 2 | FV-1 | none | 1410 | 55 | 26 |
| 3 | FV-1 | FV-1 | 2536 | 52 | 49 |
| 4 | FV-1 | FV-2 | 3311 | 54 | 62 |
| 5 | FV-1 | FV-1/FV-2 | 4319 | 50 | 86 |

*RFU = relative fluorescence units.
**Background signals were determined from control experiments that contained the probes but no target DNA.

Test Number 1 reports on the signal-generating capacity of the former assay design where the capture probe is designed to photocrosslink to the target and there are no flanking probes. The remaining tests report on the analogous signal-generating capacity of the present assay design employing varying number (i.e., 0, 1, or 2) of flanking probes.

Analysis of the signal-to-background ratios indicates that comparable sensitivity is achieved from the prior assay design (Test Number 1) as from the current assay design without flanking probes (Test Number 2). When either one or two flanking probes are incorporated into the current assay design, the sensitivity increases significantly, with the experiment using two flanking probe (Test Number 5) exhibiting an approximate 3-fold increase in sensitivity compared with that achieved from the prior assay design (Test Number 1). This result indicates that genotyping assays can now be performed using significantly less primary sample (e.g., blood), and therefore constitutes a significant improvement over the previous art.

### EXAMPLE 3

### Assay for Diagnosis of Hereditary Hemochromatosis

Background

Hereditary hemochromatosis is a relatively common autosomal recessive disorder characterized by detrimentally high absorption of iron by the body. A point mutation (G845A) in the HFE gene, resulting in a cysteine-to-tyrosine substitution at position 282 (C282Y) of the gene product, represents the primary cause of the disease. Of less clear significance to hereditary hemochromatosis is another point mutation (C187G) in the HFE gene, which results in a histidine-to-aspartate substitution at position 63 (H63D) of the gene product. Individuals who are homozygous for the C282Y mutation and, in certain instances, those who are heterozygous for the both the C282Y and H63D mutations exhibit the disease phenotype.

Assay Design

The overall assay design for genotyping either the hereditary hemochromatosis H63D or C282Y mutation is identical to that described in the Example 1 for factor V Leiden.

H63D and C282Y HFE genotyping assays employing photocrosslinking but not using flanking probes have been described in U.S. patent application Ser. No.10/093,626.

Probe Design and Synthesis

The overall probe design and synthesis is identical to that described in the previous example. The specific sequences of the probes listed below were designed to be complementary to exonic and intronic regions of the HFE gene within the context of the human genome. Additional substantive differences are noted.

Allele-Specific Capture Probes for H63D

The following two probes were designed to hybridize to either the wild-type or mutant sequence at and surrounding position 187 of the HFE gene:

| | |
|---|---|
| Capture Probe HC63-1 | |
| 3′-biotin-AXAAGATACTAGTACTCTCAG | (SEQ ID NO: 14) |

TTT-5′

| | |
|---|---|
| Capture Probe HC63-2 | |
| 3′-biotin-AXAAGATACTACTACTCTCAG | (SEQ ID NO: 15) |

TTT-5′

Capture Probe HC63-1 and Capture Probe HC63-2 target the wild-type and mutant sequences, respectively. The 3′ biotin-containing nucleotide is separated from the 3′ adenine by six spacer nucleotide units.

Flanking Probes for H63D

The following two probes were designed to hybridize and photocrosslink to the target and to form stem structures with a hybridized capture probe:

| | |
|---|---|
| Flanking Probe HC63-1 | |
| 3′-AXACTTCGAXACCCGATGCACCTACTGGTCG | (SEQ ID NO: 16) |

ACAAGCACATTT-5′

| | |
|---|---|
| Flanking Probe HC63-2 | |
| 3′-AXACGGCACACCTCGGGGCTTGAGGTACCCA | (SEQ ID NO: 17) |

XAGGTCATCTTAXA-5′

Multifluoresceinated Reporter Probes for H63D

The following seven probes were designed to hybridize and photocrosslink to the target:

| | |
|---|---|
| Reporter Probe HC63-1 | |
| 3′-T-multifluorescein- | (SEQ ID NO: 18) |

GGTCTAXACCGACGTCGACTCAGTCTCAXA-5′
Reporter Probe HC63-2

| | |
|---|---|
| 3′-T-multifluorescein- | (SEQ ID NO: 19) |

CCCACCCTAGTGTACAXGTGACAXC-5′
Reporter Probe HC63-3

| | |
|---|---|
| 3′-T-multifluorescein- | (SEQ ID NO: 20) |

GTTCCCATAXACCTCTCCCCCGGAGTGGAXG-5′
Reporter Probe HC63-4

| | |
|---|---|
| 3′-T-multifluorescein- | (SEQ ID NO: 21) |

AXAACACCCTCGTCCCTTCTCCCTTCCTTAXA-5′
Reporter Probe HC63-5

| | |
|---|---|
| 3′-T-multifluorescein- | (SEQ ID NO: 22) |

AXACCAACGTCAXTTGTTCCGACCCC-5′
Reporter Probe HC63-6

| | |
|---|---|
| 3′-T-multifluorescein- | (SEQ ID NO: 23) |

AXCCCCTACCACCTTTATCCCTGGATXA-5′
Reporter Probe HC63-7

| | |
|---|---|
| 3′-T-multifluorescein- | (SEQ ID NO: 24) |

AXGTGTGAGAGACGTGATGGAGAXG-5′

Allele-Specific Capture Probes for C282Y

The following two probes were designed to hybridize to either the wild-type or mutant sequence at and surrounding position 845 of the HFE gene:

| | |
|---|---|
| Capture Probe HC282-1 | |
| 3′-biotin-AXAATATGCACGGTCCACC | (SEQ ID NO: 25) |
| TTT-5′ | |
| Capture Probe HC282-2 | |
| 3′-biotin-AXAATATGCATGGTCCACC | (SEQ ID NO: 26) |
| TTT-5′ | |

Capture Probe HC282-1 and Capture Probe HC282-2 target the wild-type and mutant sequences, respectively. The 3′ biotin-containing nucleotide is separated from the 3′ adenine by six spacer nucleotide units.

Flanking Probes for C282Y

The following two probes were designed to hybridize and photocrosslink to the target and to form stem structures with a hybridized capture probe:

| | |
|---|---|
| Flanking Probe HC282-1 | |
| 3′-GXACCGAXATGGGGGACCCCTTCTCGTCTC | (SEQ ID NO: 27) |

TTTT-5′

| | |
|---|---|
| Flanking Probe HC282-2 | |
| 3′-AXATCGTGGGTCCGGACCTAGTCGGGGAGT | (SEQ ID NO: 28) |

XACACTAXA-5′

Multifluoresceinated Reporter Probes for C282Y

The following eleven probes were designed to hybridize and photocrosslink to the target:

| | |
|---|---|
| Reporter Probe HC282-1 | |
| 3′-T-multifluorescein- | (SEQ ID NO: 29) |

AXTCTCAGGTTAGAATCCTGTGTTTTACCACAXA-5′
Reporter Probe HC282-2

| | |
|---|---|
| 3′-T-multifluorescein- | (SEQ ID NO: 30) |

CATCGAACAAAAAXAGACTTTTCCCATAXA-5′
Reporter Probe HC282-3

| | |
|---|---|
| 3′-T-multifluorescein- | (SEQ ID NO: 31) |

AXGGAGGTTGGATATCTTCCTTCACTTTCAXG-5′
Reporter Probe HC282-4

| | |
|---|---|
| 3′-T-multifluorescein- | (SEQ ID NO: 32) |

GGAAGGAGAXAGGACAGTTCACGGAGGAXA-5′
Reporter Probe HC282-5

| | |
|---|---|
| 3′-T-multifluorescein- | (SEQ ID NO: 33) |

GTGTAGTACACTGGAGAXGTCACTGGTGAXA-5′
Reporter Probe HC282-6

| | |
|---|---|
| 3′-T-multifluorescein- | (SEQ ID NO: 34) |

AXAGCCCGGAXCTTGATGATGGGGGTCTTG-5′
Reporter Probe HC282-7

| | |
|---|---|
| 3′-T-multifluorescein- | (SEQ ID NO: 35) |

CCCACCGTTAGTTTCCGAXATTGAACGAXA-5′
Reporter Probe HC282-8

| | |
|---|---|
| 3′-T-multifluorescein- | (SEQ ID NO: 36) |

AXTACCGTCACTCTACTCCTAGACGAGAXA-5′
Reporter Probe HC282-9

| | |
|---|---|
| 3′-T-multifluorescein- | (SEQ ID NO: 37) |

AXCCCCCAXCTCTCCTCACGGACTCCTCC-5′
Reporter Probe HC282-10

| | |
|---|---|
| 3′-T-multifluorescein- | (SEQ ID NO: 38) |

AXACTGACTACTCTCGGTCCTCGACTCTTTTAXA-5′
Reporter Probe HC282-11

| | |
|---|---|
| 3′-T-multifluorescein- | (SEQ ID NO: 39) |

AXGCTTGGATTTCTGCAXAACGGGTTACCC-5′

What is claimed is:

1. A probe set for detecting a genetic polymorphism in a nucleic acid sequence of a target nucleic acid suspected of containing said polymorphism, said probe set comprising:
a) a first flanking probe comprising
i) a sequence substantially complementary to a first portion of said nucleic acid sequence, and
ii) a first side chain,
b) at least one capture probe comprising
i) a sequence substantially complementary to a second portion of said nucleic acid sequence, said second portion comprising the location of said polymorphism, said second portion being adjacent to said first portion,
ii) a second side chain substantially complementary to said first side chain, and
iii) a third side chain and
c) a second flanking probe comprising
i) a sequence substantially complementary to a third portion of said target nucleic acid sequence, said third portion being adjacent to said second portion, and
ii) a fourth side chain substantially complementary to said third side chain, wherein said first and second side chains and said third and fourth side chains non-covalently bind to form first and second stems, respectively, upon base pairing of said probes to said target nucleic acid sequence, and wherein at least one of said first and second side chains and at least one of said third and fourth side chains comprises an activatable crosslinking group, which upon activation forms a covalent cross-link with the other side chain comprising said stem, and wherein at least one of said first and second flanking probes comprises, in the sequence which is substantially complementary to its respective portion of said nucleic acid sequence, an activatable crosslinking group which upon activation forms a covalent crosslink with said respective portion.

2. A probe set according to claim 1 comprising an additional capture probe which is complementary to the normal nucleic acid sequence of said second portion lacking said polymorphism.

3. A probe set according to claim 2 additionally comprising a reporter moiety comprising a detectable label.

4. A probe set according to claim 2, wherein said crosslinking group is photoactivable.

5. A probe set according to claim 4, wherein said photoactivatable group is a coumarin, furocoumarin or psoralen.

6. A probe set according to claim 5 wherein the crosslinking compound is selected from the group consisting of coumarin, coumarin derivatives, O-(7-coumarinyl) glycerol; psoralen, psoralen derivatives, 8-methoxypsoralen, 5-methoxypsoralen; cis-benzodipyrone, cis-benzodipyrone derivatives; trans-benzodipyrone, trans-benzodipyrone derivatives; and compounds containing fused coumarin-cinnoline ring systems.

7. A probe set according to claim 6 wherein said photoactivable group is O-(7-coumarinyl) glycerol.

8. A probe set according to claim 4 wherein the polymorphism is a single nucleotide polymorphism.

9. A probe set according to claim 4 wherein the capture probes are biotinylated.

10. A probe set according to claim 8 comprising a fluoresceinated reporter molecule hybridizable to said target sequence.

11. The probe set of claim 4 wherein said polymorphism is a point mutation (G1691A) in the Factor V gene.

12. The probe set of claim 11 comprising
a first capture probe having the sequence of SEQ ID NO: 1
a second capture probe having the sequence of SEQ ID NO: 2
a first flanking probe having the sequence of SEQ ID NO: 3
a second flanking probe having the sequence of SEQ ID NO: 4
and a third flanking probe having the sequence of SEQ ID NO: 5.

13. The probe set of claim 12 comprising a fluoresceinated reporter molecule hybridizable to said target sequence.

14. The probe set according to claim 13 comprising a reporter probe selected from the group consisting of SEQ ID NO: 6, SEQ ID NO: 7, SEQ ID NO: 8, SEQ ID NO: 9, SEQ ID NO: 10, SEQ ID NO: 11, and SEQ ID NO: 12.

15. The probe set of claim 4 wherein said polymorphism is a point mutation (C187G) in the HFE gene.

16. The probe set of claim 15 comprising
a first capture probe having the sequence of SEQ ID NO: 14
a second capture probe having the sequence of SEQ ID NO: 15
a first flanking probe having the sequence of SEQ ID NO: 16
and a second flanking probe having the sequence of SEQ ID NO: 17.

17. The probe set of claim 16 comprising a fluoresceinated reporter molecule hybridizable to said target sequence.

18. The probe set according to claim 17 comprising a reporter probe selected from the group consisting of SEQ ID NO.: 18, SEQ ID NO.: 19, SEQ ID NO.: 20, SEQ ID NO.: 21, SEQ ID NO.: 22, SEQ ID NO.: 23, and SEQ ID NO.: 24.

19. The probe set of claim 4 wherein said polymorphism is a point mutation (G845A) in the HFE gene.

20. The probe set of claim 19 comprising
a first capture probe having the sequence of SEQ ID NO: 25
a second capture probe having the sequence of SEQ ID NO: 26
a first flanking probe having the sequence of SEQ ID NO: 27
and a second flanking probe having the sequence of SEQ ID NO: 28.

21. The probe set according to claim 20 comprising a fluoresceinated reporter probe hybridizable to said target sequence.

22. The probe set according to claim 21 comprising a reporter probe selected from the group consisting of SEQ ID NO.:29, SEQ ID NO.:30, SEQ ID NO.:31, SEQ ID NO.:32, SEQ ID NO.:33, SEQ ID NO.:34, SEQ ID NO.:35, SEQ ID NO.:36, SEQ ID NO.:37, SEQ ID NO.:38, and SEQ ID NO.:39.

23. A method for detecting a genetic polymorphism in a nucleic acid sequence of a target nucleic acid suspected of containing said polymorphism, said method comprising:
combining, in a hybridizing medium, a nucleic acid sample comprising said target and a plurality of probes under hybridizing conditions for a time sufficient for said target and said probes to hybridize, wherein said plurality of probes comprises
a) a first flanking probe comprising
i) a sequence substantially complementary to a first portion of said nucleic acid sequence, and
ii) a first side chain,
b) at least one capture probe comprising
i) a sequence substantially complementary to a second portion of said nucleic acid sequence, said second portion comprising the location of said polymorphism, said second portion being adjacent to said first portion,
ii) a second side chain substantially complementary to said first side chain, and
iii) a third side chain and
c) a second flanking probe comprising
i) a sequence substantially complementary to a third portion of said target nucleic acid sequence, said third portion being adjacent to said second portion, and
ii) a fourth side chain substantially complementary to said third side chain, wherein said first and second side chains and said third and fourth side chains non-covalently bind to form first and second stems, respectively, upon base pairing of said probes to said target nucleic acid sequence, and wherein at least one of said first and second side chains and at least one of said third and fourth side chains comprises an activatable crosslinking group, which upon activation forms a covalent cross-link with the other side chain comprising said stem, and wherein at least one of said first and second flanking probes comprises, in the sequence which is substantially complementary to its respective portion of said nucleic acid sequence, an activatable crosslinking group which upon activation forms a covalent crosslink with said respective portion comparing the degree of hybridization of said capture probe to said sequence portion containing said polymorphism to the hybridization of a capture probe to said target sequence lacking said polymorphism whereby the polymorphism is determined.

24. A method according to claim 23 comprising an additional capture probe which is complementary to the normal nucleic acid sequence of said second portion lacking said polymorphism.

25. A method according to claim 24 additionally comprising a reporter moiety comprising a detectable label.

26. A method according to claim 25, wherein said crosslinking group is photoactivable.

27. A method according to claim 26, wherein said photoactivatable group is a coumarin, furocoumarin or psoralen.

28. A method according to claim 27 wherein the crosslinking compound is selected from the group consisting of coumarin, coumarin derivatives, O-(7-coumarinyl) glycerol; psoralen, psoralen derivatives, 8-methoxypsoralen, 5-methoxypsoralen; cis-benzodipyrone, cis-benzodipyrone derivatives; trans-benzodipyrone, trans-benzodipyrone derivatives; and compounds containing fused coumarin-cinnoline ring systems.

29. A method according to claim 28 wherein said photoactivable group is O-(7-coumarinyl) glycerol.

30. A method according to claim 26 wherein the polymorphism is a single nucleotide polymorphism.

31. A method according to claim 26 wherein the capture probes are biotinylated.

32. A method according to claim 30 comprising a fluoresceinated reporter molecule hybridizable to said target sequence.

33. The method of claim 26 wherein said polymorphism is a point mutation (G1691A) in the Factor V gene.

34. The method of claim 33 comprising
a first capture probe having the sequence of SEQ ID NO: 1
a second capture probe having the sequence of SEQ ID NO: 2
a first flanking probe having the sequence of SEQ ID NO: 3
a second flanking probe having the sequence of SEQ ID NO: 4
and a third flanking probe having the sequence of SEQ ID NO: 5.

35. The method of claim 34 comprising a fluoresceinated reporter molecule hybridizable to said target sequence.

36. The method according to claim 35 comprising a reporter probe selected from the group consisting of SEQ ID NO: 6, SEQ ID NO: 7, SEQ ID NO: 8, SEQ ID NO: 9, SEQ ID NO: 10, SEQ ID NO: 11, and SEQ ID NO: 12.

37. The method of claim 26 wherein said polymorphism is a point mutation (C187G) in the HFE gene.

38. The method of claim 37 comprising
a first capture probe having the sequence of SEQ ID NO: 14
a second capture probe having the sequence of SEQ ID NO: 15
a first flanking probe having the sequence of SEQ ID NO: 16
and a second flanking probe having the sequence of SEQ ID NO: 17.

39. The method of claim 38 comprising a fluoresceinated reporter molecule hybridizable to said target sequence.

40. The method according to claim 39 comprising a reporter probe selected from the group consisting of SEQ ID NO.: 18, SEQ ID NO.: 19, SEQ ID NO.: 20, SEQ ID NO.: 21, SEQ ID NO.: 22, SEQ ID NO.: 23, and SEQ ID NO.: 24.

41. The method of claim 26 wherein said polymorphism is a point mutation (G845A) in the HFE gene.

42. The method of claim 41 comprising
a first capture probe having the sequence of SEQ ID NO: 25
a second capture probe having the sequence of SEQ ID NO: 26
a first flanking probe having the sequence of SEQ ID NO: 27
and a second flanking probe having the sequence of SEQ ID NO: 28.

43. The method according to claim 42 comprising a fluoresceinated reporter probe hybridizable to said target sequence.

44. The method according to claim 43 comprising a reporter probe selected from the group consisting of SEQ ID NO.:29, SEQ ID NO.:30, SEQ ID NO.:31, SEQ ID NO.:32, SEQ ID NO.:33, SEQ ID NO.:34, SEQ ID NO.:35, SEQ ID NO.:36, SEQ ID NO.:37, SEQ ID NO.:38, and SEQ ID NO.:39.

45. The method of claim 23 wherein the probes are comprised of ribonucleic acid.

46. The method of claim 23 wherein the probes are comprised of deoxyribonucleic acid.
